(19) United States
(12) Patent Application Publication
(10) Pub. No.: US 2019/0183408 A1
(43) Pub. Date: Jun. 20, 2019

(54) BREAST SIZE (VOLUME) MEASUREMENT APPARATUS

(71) Applicant: Mustafa TEZCAN, Istanbul (TR)

(72) Inventor: Mustafa TEZCAN, Istanbul (TR)

(21) Appl. No.: 16/322,932

(22) PCT Filed: Nov. 16, 2016
(86) PCT No.: PCT/TR2016/050447

§ 371 Date: Feb. 2, 2019

(30) Foreign Application Priority Data
Oct. 5, 2016 (TR) 2016/13969

(51) Int. Cl. A61B 5/00; A61B 5/107; A61B 90/17

(52) U.S. Cl. CPC A61B 5/4312; A61B 5/1073; A61B 2017/00796; A61B 90/17; A61B 5/708

(57) ABSTRACT

A breast volume measurement apparatus includes a volume measurement section, a nipple spacing, an opening for intervention, a plurality of tissue control gaps, and a plurality of fixation channels. The breast volume measurement apparatus is developed in order to ensure symmetry between two breasts during therapeutic or cosmetic breast surgery and to guide the surgeon in terms of breast size reduction ratios.

### CROSS REFERENCE TO THE RELATED APPLICATIONS

This application is the national phase entry of International Application No. PCT/TR2016/050447, filed on Nov. 16, 2016, which is based upon and claims priority to Turkish Patent Application No. 2016/13969, filed on Oct. 5, 2016, the entire contents of which are incorporated herein by reference.

### TECHNICAL FIELD

This invention is a breast volume measurement apparatus which was developed in order to ensure symmetry between two breasts during therapeutic or cosmetic breast surgery and to guide the surgeon in terms of breast size reduction ratios.

### BACKGROUND

Currently, women who wish to undergo breast surgery due to various reasons, therapeutic or cosmetic (breast reduction, augmentation or correction of deformation in the breast tissue etc.), obtain the desired appearance or medical outcome thanks to such surgical operations. However, surgeons currently conduct the necessary pre-operational measurements prior to breast surgery with the patient standing afoot, and there is lack of a proper apparatus for measurement of breast volume during the procedure itself, when the patient remains in recumbent position and measurements vary. Additionally, as tissue integrity is disturbed during an operation, pre-operational marking and measurement cannot provide the full effect intended. This leads to the surgeon essentially relying on one's own visual acuity as well as experience and skill in ensuring the desired symmetry between the two breasts. Considering specific issues such as carelessness, absent-mindedness and exhaustion, this situation where breast symmetry depends on the operating surgeon causes a certain risks, rendering some operations ineffective in terms of obtaining the desired symmetrical outcomes due to an auxiliary apparatus not being used during operation. Furthermore, due to the fact that there is currently no available apparatus to be used for the purpose grasping the entire breast and preventing tissues from slopping over, an additional supporting instrument may be needed.

### Summary

The purpose of the invention is to enable measurement of breast sizes prior to and during cosmetic breast surgeries, allowing the surgeon to obtain the desired symmetrical balance between the breasts.

The purpose of this invention is to allow the surgeon to grasp the entire breast, essentially preventing the breast from slopping over and eliminating the need for an additional supporting instrument during surgery.

The purpose of this invention is to allow breast measurement during surgery while the patient is in recumbent position, facilitating the task undertaken by the surgery and the surgical assistant.

The purpose of this invention is to facilitate, in cases where a breast prosthesis is to be used, making a decision on prosthesis size prior to and during surgical operation.

### BRIEF DESCRIPTION OF THE DRAWINGS

FIG. 1 is a perspective view of the present invention;

FIG. 2 is a top view the FIG. 1; and

FIG. 3 is a diagram of a breast size (volume) measurement apparatus in a using state.

Numbers found on the figures describe the following:
1. volume measurement section (1)
2. nipple spacing
3. opening for intervention
4. tissue control gaps
5. suture fixation channels

### DETAILED DESCRIPTION

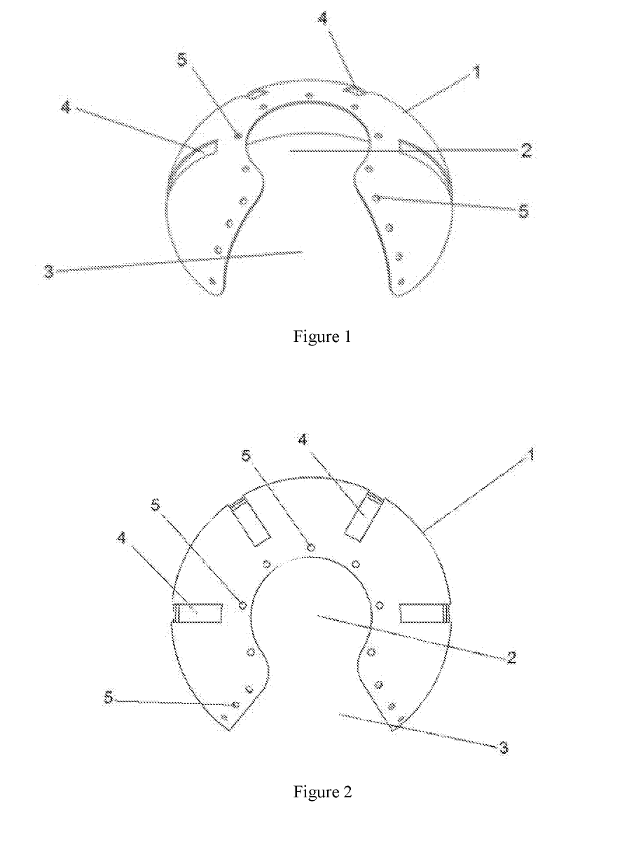

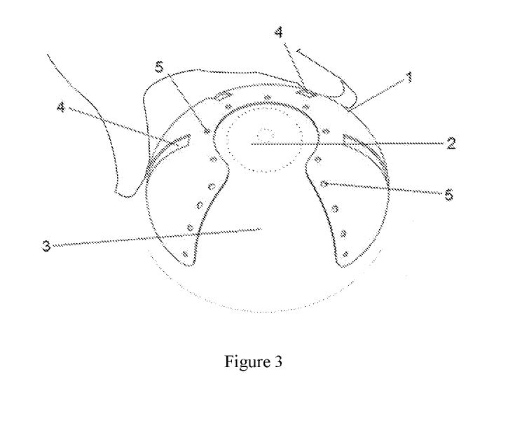

As shown in FIG. 1 and FIG. 2, a breast size (volume) measurement apparatus of the present invention includes a dome-like volume measurement section (1) made of semi-flexible metal or plastic derivatives. Using the nipple spacing (2), the surgeon may ensure that the patient's nipple is in this area, positioning the breast correctly. On the lateral surface of the volume measurement section (1), there is an opening for intervention (3) next to the nipple spacing (2). This opening for intervention (3) provides the volume measurement section (1) with a certain level of flexibility and allows necessary tissue adjustments etc. As shown in FIG. 3, the volume measurement section (1) features control gaps (4), which enable easier handling of the volume measurement section (1) as well as observing the breast tissue without it slopping over during surgery. The invention also features fixation channels (5) surrounding the nipple spacing (2) and the opening for intervention (3) above the volume measurement section (1). These fixation channels (5) allow fixation of the darker portion of the breast (nipple areola) to the invention using sutures, if deemed necessary.

The invention roughly plays the role of a mould during surgery, and thanks to its semi-flexible structure, it assumes the size/shape of the measured breast. This way, it can be placed on the other breast, which will be operated on, and both breasts can be symmetrically adjusted to each other after the surgeon performs the same operation as the first breast on the second one.

The lateral surface length of the invention, which extends from the dome-like volume measurement section (1) and the nipple spacing (2) to the base, gradually grows narrower to a certain extent as the opening for intervention is approached from the lateral side. This facilitates flexing of the dome-like volume measurement section (1). If desired, the lateral surface length, which extends from the dome-like volume measurement section (1) and the nipple spacing (2) to the base, may be redesigned so that it does not narrow down as the opening for intervention is approached from the lateral side.

What is claimed is:

1. A breast volume measurement apparatus, comprising:
a volume measurement section, wherein the volume measurement section is made of a semi-flexible metal or a plastic derivative;
a nipple spacing, wherein the nipple spacing is located in a middle of the volume measurement section, and configured to position a breast by placing a nipple in the nipple spacing;
an opening for intervention, wherein the opening for intervention is on a lateral surface of the volume measurement section and next to the nipple spacing, and the opening for intervention provides the volume measurement section with a level of flexibility;
a plurality of tissue control gaps, wherein the plurality of tissue control gaps are on the lateral surface of the volume measurement section, and the plurality of tissue control gaps facilitate a handling of the volume measurement section and prevent a breast tissue from slopping over during a surgery; and
a plurality of fixation channels, wherein the plurality of fixation channels surround the nipple spacing and the opening for intervention above the volume measurement section and allow a fixation of a darker portion of the breast (nipple areola) to the volume measurement section using sutures.

2. The breast volume measurement apparatus of claim 1, wherein the lateral surface of the volume measurement section is formed by extending from the volume measurement section and the nipple spacing to a base, and a length of the lateral surface gradually grows narrower as the opening for intervention is approached from a lateral side.
